(19) United States
(12) Patent Application Publication
(10) Pub. No.: US 2009/0028962 A1
(43) Pub. Date: Jan. 29, 2009

(54) Methods and Compositions for Enhancing Iron Absorption

(75) Inventors: Jonathan David Bortz, Saint Louis, MO (US); Mitchell Kirschner, Saint Louis, MO (US)

Correspondence Address: KV PHARMACEUTICAL COMPANY, 4080B WEDGEWAY COURT, EARTH CITY, MO, 63045, US

(73) Assignee: DRUGTECH CORPORATION, Wilmington, DE (US)

(21) Appl. No.: 11/793,517

(22) PCT Filed: Nov. 9, 2005
(86) PCT No.: PCT/US2005/041139

§ 371 Date: Apr. 11, 2008

Related U.S. Application Data
(63) Continuation of application No. 11/020,801, filed on Dec. 22, 2004.

(51) Int. Cl. A61K 33/26; A61K 31/375

(52) U.S. Cl. 424/646

(57) ABSTRACT

The present invention generally relates to methods and compositions useful in enhancing iron absorption in a patient. The methods and compositions of the present invention may be used independently to promote and/or maintain iron absorption in a patient or may be used in combination with one or more other compositions used in the treatment of one or more diseases having iron deficiency associated therewith.

### CROSS REFERENCE TO RELATED APPLICATIONS

This application claims priority as a continuation-in-part of U.S. patent application Ser. No. 11/020,801 filed Dec. 22, 2004, the entire disclosure of which is incorporated herein by reference.

### FIELD OF THE INVENTION

The present invention generally relates to methods for enhancing iron absorption in a patient. The invention is also directed generally to compositions for use in such methods to enhance iron absorption in a patient. The compositions of the present invention may be used independently to promote and/or maintain iron absorption in a patient or may be used in combination with one or more other compositions used in the treatment of one or more diseases having iron deficiency associated therewith.

### BACKGROUND OF THE INVENTION

Iron is an essential mineral for tissue growth in both humans and other animals. Although iron is normally amply available in the diet, sufficient iron is not always absorbed from food. Two proteins in the intestinal mucosal cells help the body absorb iron from food. One protein called mucosal ferritin, receives iron from the GI tract and stores it in the mucosal cells. When the body needs iron, mucosal ferritin releases some iron to another protein called mucosal transferrin. Mucosal transferrin transfers the iron to a carrier in the blood called blood transferrin, which transports iron to the rest of the body.

Iron is soluble at low pH. Thus, based on pH considerations, it appears that optimum iron absorption occurs in the acidic environment of the proximal duodenal area of the intestine. As iron consumed in the diet or through oral supplementation reaches the stomach, it may be bound to dietary substances such as phytates found in various grains. Iron bound to such dietary substances inhibits or decreases iron absorption in the small intestine. The mucosal lining of the small intestine contains fingerlike projections called “villi.” The villi are lined by cells that are formed in villi clefts and migrate toward the apices of the villi. Enterocyte cells near the apices of the villi are active absorption sites for iron. Iron absorption is inhibited in the small intestine when iron is bound to dietary substances since bound iron is unavailable for absorption by small intestine enterocyte cells.

Once iron is transported from the intestinal lumen into small intestine enterocyte cells, it forms a labile iron pool from which iron is then transported across basolateral membranes and into the blood stream. The extent of the labile iron pool regulates the amount of iron absorbed by small intestine enterocyte cells. As the labile iron pool expands, the amount of iron absorbed by small intestine enterocyte cells from the intestinal lumen and the amount of iron transported across basolateral membranes into the circulation is reduced.

The principal mechanism by which iron overload and thereby iron toxicity can be prevented, is through a very tightly regulated absorption process in which the small intestine enterocyte cells play a key role. Small intestine enterocyte cells regulate the transport and storage of iron. If iron in the labile iron pool of the small intestine enterocyte cells is not transported across the basolateral membranes, the untransported iron is lost when the enterocyte cells are sloughed off after several days. This is the chief mechanism by which the body excretes unabsorbed iron.

Iron-containing preparations have been available to treat iron deficiency, and particularly iron deficiency anemia, since the late 19^{th} century. For example, oral ferrous sulfate remains the conventional choice for dietary iron supplementation as it is considered a safe, cheap and effective means of replenishing iron stores in the vast majority of patients. However, only about 5 to 25 percent of ingested iron sulfate is absorbed. Conventional studies often extrapolated early iron absorption data over long periods of time. However, iron absorption does not remain constant over time. Iron absorption rates, regardless of the iron compound used, with or without promoters, appear to show a marked decrease in absorption after the first 20 days of daily iron supplementation. The conventionally accepted average iron absorption rate of 15 percent appears to be accurate only for iron supplementation days 1 through 20. For days 21 through 30, the average iron absorption rate of a ferrous sulfate supplement dropped to 5.1 percent in published data. See, Halberg L, Norrby A, Solvell L., “Oral Iron with Succinic Acid in the Treatment of Iron Deficiency Anemia,” Scand. J. Haematol, vol. 8, pp. 104-11 (1971). There is therefore a need for a composition that effectively enhances and/or maintains the rate of iron absorption in a patient.

### SUMMARY OF THE INVENTION

This invention is directed generally to methods for increasing iron absorption in a patient. The methods may be used independently to promote and/or maintain iron absorption in a patient or may be used in combination with one or more other methods or compositions used in the treatment of one or more diseases having iron deficiency associated therewith.

In one embodiment, the invention is directed to a method of increasing iron absorption in a patient. The method comprises administering at least two iron absorption promoters to a patient in need thereof. In a particular embodiment, at least one of the iron absorption promoters is selected to increase iron absorption within the intestinal lumen of the patient and at least one of the iron promoters is selected to increase systemic iron absorption. In another particular embodiment, a first iron absorption promoter is formulated for immediate release upon oral administration to the patient and a second iron absorption promoter is formulated for extended release upon oral administration to the patient.

In another embodiment, the invention is directed to a method of increasing iron absorption in a patient. The method comprises administering a first iron promoter comprising a compound having Vitamin C activity to a patient in need thereof; and administering a second iron promoter comprising an organic acid selected from the group consisting of succinic acid, acetic acid, citric acid, lactic acid, malic acid, glutamic acid, salts of succinic acid, salts of acetic acid, salts of citric acid, salts of lactic acid, salts of malic acid, salts of glutamic acid, derivatives of succinic acid, derivatives of acetic acid, derivatives of citric acid, derivatives of lactic acid, derivatives of malic acid, derivatives of glutamic acid, and combinations thereof to a patient in need thereof. The method is further characterized in that the second iron promoter is formulated for extended release such that less than substantially all of the second iron promoter dissolves within about 180 minutes following oral administration of the composition to a patient and substantially all of the second iron promoter dissolves in less than about 48 hours following oral administration of the composition to a patient.

This invention is also directed, in part, to compositions useful in enhancing iron absorption in a patient. In one embodiment, the composition comprises two or more iron absorption promoters wherein at least one of the iron absorption promoters is selected to increase iron absorption within the intestinal lumen and at least one of the iron absorption promoters is selected to increase systemic iron absorption when the composition is orally administered to a patient.

In another embodiment, the invention is directed to a composition for enhancing iron absorption in a patient. The composition comprises at least two iron absorption promoters wherein a first of the iron absorption promoters is formulated for immediate release when orally administered to a patient and a second of the iron absorption promoters is formulated for extended release when orally administered to a patient.

In another embodiment, the invention is directed to a composition comprising from about 5 mg to about 500 mg of a first iron absorption promoter and from about 5 mg to about 500 mg of a second iron absorption promoter. The composition is further characterized in that substantially all of the first iron absorption promoter dissolves in less than about 180 minutes following oral administration of the composition to a patient, less than substantially all of the second iron promoter dissolves within about 180 minutes following oral administration of the composition to a patient, and substantially all of the second iron promoter dissolves in less than about 48 hours following oral administration of the composition to a patient.

In another embodiment, the invention is directed to a composition for increasing iron absorption in a patient. The composition comprises a first iron promoter comprising a compound having Vitamin C activity; and a second iron promoter comprising an organic acid selected from the group consisting of succinic acid, acetic acid, citric acid, lactic acid, malic acid, glutamic acid, salts of succinic acid, salts of acetic acid, salts of citric acid, salts of lactic acid, salts of malic acid, salts of glutamic acid, derivatives of succinic acid, derivatives of acetic acid, derivatives of citric acid, derivatives of lactic acid, derivatives of malic acid, derivatives of glutamic acid, and combinations thereof. The composition is further characterized in that the second iron promoter is formulated for extended release such that less than substantially all of the second iron promoter dissolves within about 180 minutes following oral administration of the composition to a patient and substantially all of the second iron promoter dissolves in less than about 48 hours following oral administration of the composition to a patient.

In another embodiment, the invention is directed to a method of treating an iron deficiency related disease or disorder in a human by orally administering an effective amount of the compositions described herein to a human in need thereof.

Still further, the invention is directed to a pharmaceutical kit. The kit comprises a source of a first iron absorption promoter selected to increase iron absorption in the intestinal lumen upon administration to a human and a source of a second iron absorption promoter selected to increase systemic iron absorption upon administration to a human, wherein the iron absorption promoter sources are present in the kit in a therapeutically effective amount.

Further areas of applicability of the present invention will be apparent to one skilled in the art from reading this patent. It should be understood that the detailed description and specific examples, while indicating a certain, preferred embodiment of the invention, are intended for purposes of illustration only and are not intended to limit the scope of the invention.

### DETAILED DESCRIPTION

This detailed description is intended only to acquaint others skilled in the art with Applicants' invention, its principles, and its practical application so that others skilled in the art may adapt and apply the invention in its numerous forms, as they may be best suited to the requirements of a particular use. This description and its specific examples are intended for purposes of illustration only. This invention, therefore, is not limited to the embodiments described in this application, and may be variously modified.

In accordance with the present invention, Applicants have discovered that the amount of iron absorbed by a patient may be increased or enhanced by the administration of two or more iron absorption promoters. For example, without being held to a particular theory, it is has been found that synergistic effects in the amount of iron absorbed by a patient can be achieved by administering two different promoters with at least two different modes of action that are complimentary, additive or non-competitive. In particular, Applicants have demonstrated that the amount of iron absorbed by a patient can be increased by administering at least one iron absorption promoter to a patient in need thereof to enhance iron absorption in the proximal duodenal area of the intestine and at least one other iron absorption promoter to enhance systemic iron absorption.

As previously described above, pH indicators indicate that optimum iron absorption occurs in the acidic environment of the proximal duodenal area of the intestine. As iron consumed in the diet or through oral supplementation reaches the stomach, it may be bound to dietary substances such as phytates found in various grains. Iron bound to such dietary substances inhibits or decreases iron absorption in the small intestine since bound iron is unavailable for absorption by small intestine enterocyte cells. However, when an iron absorption promoter is present, the promoter competitively binds to iron, protecting the iron from phytate binding. Also, the use of an acidic iron absorption promoter (i.e., one which can act as a reducing agent) may be beneficial in maintaining favorable pH conditions in the proximal duodenal area of the intestine for optimum iron absorption.

Once iron is transported from the intestinal lumen into small intestine enterocyte cells, it forms a labile iron pool from which iron is then transported across basolateral membranes and into the blood stream. The extent of the labile iron pool regulates the amount of iron absorbed by small intestine enterocyte cells. As the labile iron pool expands, the amount of iron absorbed by small intestine enterocyte cells and the amount of iron transported across basolateral membranes is reduced. Thus, providing a second, systemic iron absorption promoter may further increase iron absorption by increasing the transfer of iron already absorbed by small intestine enterocyte cells to the basolateral cell membranes of intestinal mucosal cells.

Thus, it has been found that by administering combinations of iron absorption promoters for increasing iron absorption in the intestinal lumen (i.e., effectively “pushing” additional iron into the small intestine enterocyte cells) along with increasing systemic iron absorption (i.e., effectively “pulling” additional iron across the basolateral cell membranes), overall iron absorption in a patient can be increased. Accordingly, in one embodiment, a method of the invention comprises administering at least two iron absorption promoters to a patient in need thereof wherein at least one of the iron absorption promoters is selected to increase iron absorption within the intestinal lumen of the patient and at least one of the iron absorption promoters is selected to increase systemic iron absorption.

In another embodiment, a method of the invention comprises administering to a patient in need thereof at least two iron absorption promoters wherein a first promoter is formulated for immediate release when administered to the patient and a second promoter is formulated for extended release when administered to the patient. As used herein, an iron absorption promoter formulated for “immediate release” is an iron absorption promoter which is formulated such that substantially all of the promoter dissolves in less than about 20 minutes following oral administration to a human. In particular embodiments, substantially all of the “immediate release” promoter dissolves in less than about 15 minutes, less than about 10 minutes, or less than about 1 minute following oral administration to a human.

Likewise, an iron absorption promoter formulated for “extended release” is used herein as an iron absorption promoter which is formulated such that less than substantially all of the extended release promoter dissolves within about 20 minutes following administration to a patient and substantially all of the extended release promoter dissolves in less than about 48 hours following administration of the composition to a patient. In particular embodiments, less than substantially all of the “extended release” promoter dissolves within about 45 minutes, within about 90 minutes, or within about 180 minutes following administration of the composition to a patient.

In another embodiment, the extended release iron absorption promoter is formulated such that less than substantially all of the extended release promoter dissolves within about 8 hours following administration of the composition to a patient and substantially all of the extended release promoter dissolves in less than about 24 hours following administration of the composition to a patient.

Examples of suitable iron absorption promoters for use in the present invention include, without limitation, ascorbic acid, succinic acid, acetic acid, citric acid, lactic acid, malic acid, glutamic acid, salts of ascorbic acid, salts of succinic acid, salts of acetic acid, salts of citric acid, salts of lactic acid, salts of malic acid, salts of glutamic acid, derivatives of ascorbic acid, derivatives of succinic acid, derivatives of acetic acid, derivatives of citric acid, derivatives of lactic acid, derivatives of malic acid, derivatives of glutamic acid, compounds having Vitamin C activity, mannitol, sorbitol, xylose, inositol, fructose, sucrose, lactose, glucose, calcium, copper, sodium molybdate, amino acids and combinations thereof.

In one embodiment, the lumenal or first iron absorption promoter which is formulated for immediate release comprises ascorbic acid or another compound having Vitamin C activity. As used. herein, “compounds having Vitamin C activity” include Vitamin C (i.e., L-ascorbic acid) and any derivative or metabolite of ascorbic acid that exhibits ascorbic activity as determined by a standard iodine titration test. Suitable derivatives of ascorbic acid include, for example, oxidation products such as dehydroascorbic acid and edible salts of ascorbic acid such as calcium ascorbate, sodium ascorbate, magnesium ascorbate, potassium ascorbate and zinc ascorbate. Suitable metabolites of ascorbic acid include, for example, aldo-lactones and edible salts of aldonic acids including L-threonic acid, L-xylonic acid and L-lyxonic acid.

A suitable form of ascorbic acid for use in the present invention is a buffered Vitamin C preparation such as calcium ascorbate. A particular form of calcium ascorbate is Ester C® (commercially available from Zila Nutraceuticals, Inc., Prescott, Ariz.), as disclosed in U.S. Pat. Nos. 4,822,816 and 5,070,085, both of which are incorporated herein by reference in their entirety.

In another embodiment, the systemic or second iron absorption promoter which is formulated for extended release comprises an organic acid selected from the group consisting of succinic acid, acetic acid, citric acid, lactic acid, malic acid, glutamic acid, salts of succinic acid, salts of acetic acid, salts of citric acid, salts of lactic acid, salts of malic acid, salts of glutamic acid, derivatives of succinic acid, derivatives of acetic acid, derivatives of citric acid, derivatives of lactic acid, derivatives of malic acid, derivatives of glutamic acid and combinations thereof.

In still another embodiment, the lumenal or first iron absorption promoter comprises ascorbic acid and the systemic or second iron absorption promoter comprises succinic acid. It is important to note that ascorbic acid has been found to enhance gastrointestinal iron absorption only upon oral administration. Gastrointestinal iron absorption is not increased by intravenous administration of ascorbic acid. However, succinic acid has been found to enhance gastrointestinal iron absorption when administered both orally and parenterally. Accordingly, in one embodiment, a method of the invention comprises orally administering a lumenal or first iron absorption promoter for immediate release to a patient and parenterally administering a systemic or second iron absorption promoter to the patient.

The iron absorption promoters of the compositions of the present invention are independently provided in an effective amount of about 5 mg to about 500 mg, more preferably about 100 mg to about 500 mg and most preferably about 150 mg to about 500 mg per dosage, to promote iron absorption as discussed in still greater detail below. In the case of products developed for pediatric use, the effective amount of the iron absorption promoters would be reduced to levels considered safe for infants and children. An effective amount of one or more forms of an organic acid or combinations thereof for pediatric applications may be as low as about 0.50 mg of iron absorption promoter per kilogram of body weight per dosage.

When the lumenal and systemic iron absorption promoters are provided in a single composition or pharmaceutical kit, the molar ratio of lumenal promoter to systemic promoter is typically from 1 to 1.5. Thus, a particular example of a composition of the present invention includes about 25 mg to about 500 mg of one or more forms of ascorbic acid as the lumenal promoter and about 25 mg to about 500 mg of one or more forms of succinic acid as the systemic promoter per dosage.

The methods of the present invention may further comprise the administration of one or more sources of iron to the patient. Likewise, the compositions of the invention may further comprise at least one source of iron. Suitable sources of iron can include any pharmaceutically acceptable iron compound, for example, an iron (II) salt or an iron (III) salt, or a metallic form of iron (e.g. carbonyl iron). Examples of suitable iron compounds include, without limitation, ferrous fumarate, ferrous sulfate, ferrous folate, an iron dextran, ferric oxyhydroxide dextran, a chitosan derivative of iron, an oligosaccharide derivative of iron, ferrous acetyl salicylate, ferrous gluconate, ferrous diphosphate, carbonyl iron, ferric orthophosphate, ferrous glycine sulfate, ferrous chloride, ferrous ammonium citrate, ferric ammonium citrate, ferric ammonium tartrate, ferric phosphate, ferric potassium tartrate, ferric albuminate, ferric cacodylate, ferric hydroxide, ferric pyrophosphate, ferric quinine citrate, ferric valerate, saccharated iron oxide, iron oxide, ferric chloride, ferrous iodide, ferrous nitrate, ferrous glycerophosphate, ferrous formate, an amino acid and iron salt, an iron salt of a protein hydrolysate, ferrous lactate, ferrous tartrate, ferrous succinate, ferrous glutamate, ferrous citrate, ferrous pyrophosphate, ferrous cholinisocitrate, ferrous carbonate, an iron-sugar-carboxylate complex, ferrous sucrate-malate, ferrous sucrate citrate, ferrous fructate-citrate, ferrous sucrate-ascorbate, and ferrous fructate-ascorbate.

Other suitable forms of iron for purposes of the present invention include for example but are not limited to soluble iron salts, slightly soluble iron salts, insoluble iron salts, chelated iron, iron complexes, non-reactive iron such as carbonyl iron and reduced iron, and combinations thereof.

Preferred chelated iron complexes are disclosed in U.S. Pat. Nos. 4,599,152 and 4,830,716, which are each incorporated herein by reference in their entirety.

Examples of suitable soluble iron salts include but are not limited to ferric hypophosphite, ferric albuminate, ferric chloride, ferric citrate, ferric oxide saccharate, ferric ammonium citrate, ferrous chloride, ferrous gluconate, ferrous iodide, ferrous sulfate, ferrous lactate, ferrous fumarate, haeme, ferric trisglycinate, ferrous bisglycinate, ferric nitrate, ferrous hydroxide saccharate, ferric sulfate, ferric gluconate, ferric aspartate, ferrous sulfate heptahydrate, ferrous phosphate, ferric ascorbate, ferrous formate, ferrous acetate, ferrous malate, ferrous glutamate, ferrous cholinisocitrate, ferroglycine sulfate, ferric oxide hydrate, ferric pyrophosphate soluble, ferric hydroxide saccharate, ferric manganese saccharate, ferric subsulfate, ferric ammonium sulfate, ferrous ammonium sulfate, ferric sesquichloride, ferric choline citrate, ferric manganese citrate, ferric quinine citrate, ferric sodium citrate, ferric sodium edetate, ferric formate, ferric ammonium oxalate, ferric potassium oxalate, ferric sodium oxalate, ferric peptonate, ferric manganese peptonate, other pharmaceutically acceptable iron salts, and combinations thereof.

Examples of suitable slightly soluble iron salts include but are not limited to ferric acetate, ferric fluoride, ferric phosphate, ferric pyrophosphate, ferrous pyrophosphate, ferrous carbonate saccharated, ferrous carbonate mass, ferrous succinate, ferrous citrate, ferrous tartrate, ferric fumarate, ferric succinate, ferrous hydroxide, ferrous nitrate, ferrous carbonate, ferric sodium pyrophosphate, ferric tartrate, ferric potassium tartrate, ferric subcarbonate, ferric glycerophosphate, ferric saccharate, ferric hydroxide saccharate, ferric manganese saccharate, ferrous ammonium sulfate, other pharmaceutically acceptable iron salts, and combinations thereof.

Examples of suitable insoluble iron salts include but are not limited to ferric sodium pyrophosphate, ferrous carbonate, ferric hydroxide, ferrous oxide, ferric oxyhydroxide, ferrous oxalate, other pharmaceutically acceptable iron salts and combinations thereof.

Examples of suitable iron complexes include but are not limited to polysaccharide-iron complex, methylidine-iron complex, ethylenediaminetetraacetic acid (EDTA)-iron complex, phenanthrolene iron complex, p-toluidine iron complex, ferrous saccharate complex, ferrlecit, ferrous gluconate complex, ferrum vitis, ferrous hydroxide saccharate complex, iron-arene sandwich complexes, acetylacetone iron complex salt, iron-dextran complex, iron-dextrin complex, iron-sorbitol-citric acid complex, saccharated iron oxide, ferrous fumarate complex, iron porphyrin complex, iron phtalocyamine complex, iron cyclam complex, dithiocarboxy-iron complex, desferrioxamine-iron complex, bleomycin-iron complex, ferrozine-iron complex, iron perhaloporphyrin complex, alkylenediamine-N,N-disuccinic acid iron(III) complex, hydroxypyridone-iron(III) complex, aminoglycoside-iron complex, transferrin-iron complex, iron thiocyanate complex, iron complex cyanides, porphyrinato iron(III) complex, polyaminopolycarbonate iron complexes, dithiocarbamate iron complex, adriamycin iron complex, anthracycline-iron complex, N-methyl-D-glucamine dithiocarbamate (MGD)-iron complex, ferrioxamine B, ferrous citrate complex, ferrous sulfate complex, ferric gluconate complex, ferrous succinate complex, polyglucopyranosyl iron complex, polyaminodisuccinic acid iron complex, biliverdin-iron complex, deferiprone iron complex, ferric oxyhydride-dextran complex, dinitrosyl dithiolato iron complex, iron lactoferrin complexes, 1,3-ethylenediaminetetraacetic acid (EDTA) ferric complex salts, diethylenetriaminepentaacetic acid iron complex salts, cyclohexanediaminetetraacetic acid iron complex salts, methyliminodiacetic acid iron complex salts, glycol ether diaminetetraacetic acid iron complex salts, ferric hydroxypyrone complexes, ferric succinate complex, ferric chloride complex, ferric glycine sulfate complex, ferric aspartate complex, sodium ferrous gluconate complex, ferrous hydroxide polymaltose complex, glycine-aspartic acid complexes, other pharmaceutically acceptable iron complexes and combinations thereof.

Suitable forms of iron for purposes of the present invention also include iron compounds designated as “slow dissolving” or “slow acting” and iron compounds designated as “fast dissolving” or “fast acting.” Compositions of the present invention may optionally include at least two iron compounds, e.g., at least one iron compound designated slow acting and at least one iron compound designated as fast acting. The use of two such differing iron compounds in a formulation is disclosed in U.S. Pat. No. 6,521,247, which is incorporated herein by reference in its entirety. Compositions of the present invention may also include extended release iron compounds and/or controlled release iron compounds.

In one embodiment, the composition of the invention comprises a bis-glycine chelate of iron, for example, Ferrochel™ (commercially available from Albion International, Inc., Clearfield, Utah). While the bis-glycine chelate of iron is preferred, any number of suitable chelates may be used. For example, amino acid chelates are becoming well accepted as a means of increasing the metal content in biological tissues of man, animals and plants. Amino acid chelates are products resulting from the reaction of a polypeptide, dipeptide or naturally occurring alpha amino acid with a metal ion having a valence of two or more. The alpha amino acid and metal ion form a ring structure wherein the positive electrical charges of the metal ion are neutralized by the electrons of the carboxylate or free amino groups of the alpha amino acid. Although the term amino acid as used herein refers only to products obtainable through protein hydrolysis, synthetically produced amino acids are not to be excluded provided they are the same as those obtained through protein hydrolysis. Accordingly, protein hydrolysates such as polypeptides, dipeptides and naturally occurring alpha amino acids are collectively referred to as amino acids. Additional suitable amino acid chelates include for example but are not limited to ethylenediaminetetraacetic acid (EDTA), monohydroxyethylethylenediaminetriacetic acid, diethylenetriaminepentaacetic acid, monohydroxyethyldiglycine and dihydroxyethylglycine.

Compositions of the present invention include one or more forms of iron in an effective amount of from about 5 mg to about 500 mg, more preferably from about 50 mg to about 500 mg and most preferably from about 150 mg to about 500 mg per dosage. In the case of products developed for pediatric use, an effective amount of iron would be greatly reduced to levels considered safe for infants and children. An effective amount of one or more forms of iron for pediatric applications may be as low as about 0.5 mg of iron per kilogram of body weight per dosage.

Additionally, compositions of the present invention may be administered in combination with group B Vitamins, and/or laxatives, and/or anti-emetic agents, and/or birth control agents, and/or one or more other compositions used in the treatment of one or more diseases having iron deficiency associated therewith.

Optionally, the iron absorption promoters, or any other of the individual components of the compositions of the present invention may be formulated as coated or treated for controlled release to optimize absorption. In coating or treating the components, components could be coated or treated with the same coating or treatment, or could be coated individually with one or more differing coatings or treatments. Likewise one or more components could be coated or treated and combined with one or more components that are uncoated or untreated. Such coating or treatment variations are useful to manipulate and control the release of each component so as to optimize absorption.

A dosage of one or more compositions of the present invention may be manufactured in one or more dosage forms such as for example but not limited to a tablet, caplet, capsule, gel capsule, chew tablet, lozenge and troche, nutritional bar or food item, soft chew, reconstitutable powder or shake, sprinkle, semi-solid sachet or the like. Any tablet dosage form may be either chewable or compressed. The preferred solid dosage form for purposes of the present invention is a capsule or tablet. However, compositions of the present invention could likewise be incorporated into a food product or a powder for mixing with a liquid. Although any number of suitable dosage forms can be used to administer compositions of the present invention, preferred dosage forms include a single capsule, two capsules or one capsule and one caplet or tablet.

Compositions of the present invention can not only be provided in various dosage forms but can also be administered in accordance with various dosage regimens as described in more detail below. For example, a dosage of one or more compositions of the present invention may be administered as one more dosage units and in one or more dosage forms. Further, such dosage forms can be for enteral and/or parenteral administration such as but not limited to oral, intraperitoneal, intravenous, subcutaneous, transcutaneous or intramuscular routes of administration.

For example, in one embodiment, the present invention is directed to a pharmaceutical kit comprising a source of a first iron absorption promoter selected to increase iron absorption in the intestinal lumen upon administration to a human, and a source of a second iron absorption promoter selected to increase systemic iron absorption upon administration to a human, wherein the iron absorption promoter sources are present in the pharmaceutical kit in a therapeutically effective amount.

In a particular embodiment, the pharmaceutical kit comprises a source of a compound having Vitamin C activity as the first iron absorption promoter and a source of an organic acid selected from the group consisting of succinic acid, acetic acid, citric acid, lactic acid, malic acid, glutamic acid, salts of succinic acid, salts of acetic acid, salts of citric acid, salts of lactic acid, salts of malic acid, salts of glutamic acid, derivatives of succinic acid, derivatives of acetic acid, derivatives of citric acid, derivatives of lactic acid, derivatives of malic acid, derivatives of glutamic acid, and combinations thereof as the second iron absorption promoter. In a particular embodiment, the kit comprises a source of a compound having Vitamin C activity selected from the group consisting of L-ascorbic acid, calcium ascorbate, sodium ascorbate, magnesium ascorbate, potassium ascorbate, zinc ascorbate, L-threonic acid, L-xylonic acid and L-lyxonic acid and a source of succinic acid as the second iron absorption promoter.

In a still further embodiment, the pharmaceutical kits of the present invention further comprises a therapeutically effective amount of one or more elemental sources of iron as described above. In such an embodiment, the kit may comprise at least 3 separate unit dosages including a unit dosage comprising the source of the first iron absorption promoter, a unit dosage comprising the source of the second iron absorption promoter and a unit dosage comprising a source of iron.

In a still further embodiment, the teachings of the present disclosure provide for treatment regimens using the compositions and kits described herein for promoting and/or maintaining iron absorption in a patient. For example, such a treatment regimen may comprise an alternating day administration over one to thirty days wherein a source of iron and a first iron absorption promoter is administered on a first day followed by a source of iron and a second iron absorption promoter administered on a second day. Another alternating one to thirty day treatment regimen may comprise a source of iron and first iron absorption promoter administered on a first day followed by a second iron absorption promoter administered on a second day. Still another exemplary alternating treatment regimen may comprise a first iron absorption promoter administered on a first day, a source of iron administered on a second day, and a second iron absorption promoter administered on a third day.

The compositions and kits of the present invention may be used independently to promote and/or maintain iron absorption or used in combination with one or more other compositions used in the treatment of one or more diseases having iron deficiency associated therewith. Such diseases or conditions associated with iron deficiency include for example but are not limited to gastro-intestinal diseases or conditions that cause blood loss such as for example but not limited to infectious parasites such as hookworms, regular use of non-steroidal anti-inflammatory drugs, steroids and/or aspirin, peptic ulcer disease, gastritis, colon cancer, polyps and inflammatory bowel disease, gastro-intestinal diseases or conditions that cause decreased absorption of iron such as for example but not limited to tropical sprue, celiac disease, autoimmune diseases, gastrectomy, gastric bypass, vagotomy and diseases requiring therapy with proton pump inhibitors and H₂ antagonists, neurological diseases or conditions such as for example but not limited to restless leg syndrome, chronic fatigue, cognitive deficiencies and neuro-developmental deficiencies, physiological conditions such as for example but not limited to sports, menses, lactation, pregnancy and surgery, infectious diseases such as for example but not limited to HIV/AIDS and malaria, chronic diseases such as for example but not limited to cancer, rheumatoid arthritis and chronic renal failure and heavy metal poisoning such as for example but not limited to lead, mercury, cadmium and arsenic.

In another embodiment of the present invention, the iron absorption promoters are provided along with a nutritional iron supplement for blood-iron concentration maintenance purposes. An illustrative composition for such blood-iron concentration maintenance includes from about 10 mg to about 70 mg iron, from about 5 mg to about 150 mg succinic acid and from about 5 mg to about 200 mg ascorbic acid per dosage. Compositions for blood-iron concentration maintenance are useful for humans or other animals that are mildly iron deficient, post iron therapy, or are part of an “at risk” population, such as for example but not limited to regular blood donors.

The iron absorption promoters may also be provided along with nutritional or dietary iron supplement compositions for therapeutic purposes. In one embodiment, the iron absorption promoters are provided along with a dietary iron supplement composition in a therapeutic regimen. An exemplary three-week therapeutic regimen comprises administering a lumenal iron absorption promoter such as Vitamin C along with iron in Week 1. The regimen for Week 2 comprises administering the lumenal iron absorption promoter with a systemic iron absorption promoter such as succinic acid and optionally iron. Week 3 comprises administering the lumenal iron absorption promoter and the systemic iron absorption promoter.

The amounts of iron absorption promoters provided as part of a therapeutic regimen may vary widely. For example, various amounts of a first promoter to increase lumenal iron absorption along with various amounts of a second promoter to increase systemic iron absorption can be employed depending on the individual patient and/or iron deficiency condition being treated. For example, in one embodiment, a regimen for enhancing iron absorption involves a one-month treatment regimen comprising the administration of ascorbic acid as a first iron absorption promoter and succinic acid as a second iron absorption promoter. Exemplary amounts of each promoter for administration include:

| | Ascorbic acid per dosage | Succinic acid per dosage |
|---|---|---|
| Week 1 | 100 mg | 0 mg |
| Week 2 | 200 mg | 100-150 mg |
| Week 3 | 100 mg | 150 mg |
| Week 4 | 0 mg | 200 mg |

In another embodiment, the iron absorption promoters are provided as part of a nutritional or dietary iron supplement composition for therapeutic purposes. An illustrative composition for therapeutic iron supplementation comprises 70 mg iron, 150 mg succinic acid and 200 mg ascorbic acid per dosage. This therapeutic nutritional or dietary supplement composition is useful for iron deficient humans or other animals. Such therapeutic compositions are preferably supplied in a once daily, 21-day calendar pack for monthly iron supplementation therapy. In such a case, absorbed iron provides sufficient iron for approximately 1 g per month of hemoglobin regeneration as well as iron for iron store repletion. It is preferable that iron supplementation be discontinued for at least a week following administration of the 21-day pack to allow absorption rates to remain high during administration weeks, thus optimizing the same. However, for women in their childbearing years, compositions of the present invention may be administered for seven days during menstruation to replenish lost iron, followed by discontinued iron supplementation for 21 days.

In yet another embodiment, the iron absorption promoters are provided as part of a nutritional or dietary iron supplement composition for therapeutic purposes. An illustrative composition for therapeutic iron supplementation includes 150 mg of a bis-glycine chelate of iron, 150 mg succinic acid and 200 mg ascorbic acid per dosage. This therapeutic nutritional or dietary supplement composition is useful for iron deficient humans or other animals. Such therapeutic compositions are preferably supplied in a three times daily, 21-day calendar pack for monthly iron supplementation therapy. In such a case, absorbed iron could provide approximately 3 g per month of iron for hemoglobin regeneration and iron store repletion. As with all the nutritional or dietary supplement compositions of the present invention, it is preferable that the iron supplementation be discontinued for at least a week following administration of the 21-day pack to allow iron absorption rates to remain at their peak during administration weeks.

The above description of the invention is merely exemplary in nature and, thus, variations that do not depart from the gist of the invention are intended to be within the scope of the invention. Such variations are not to be regarded as a departure from the spirit and scope of the invention

### EXAMPLES

### Example 1

A composition including a bis-glycine chelate of iron (70 mg, FERROCHEL), ferrous fumarate iron (81 mg), and ascorbic acid (200 mg) in an immediate release dosage form and succinic acid (150 mg) in an extended release dosage form.

### Example 2

A composition including elemental iron (151 mg), succinic acid (150 mg), ascorbic acid (60 mg), folic acid (1 mg) and Vitamin B₁₂ (10 μg).

### Example 3

A composition including elemental iron (151 mg), succinic acid (150 mg), ascorbic acid (200 mg), folic acid (1 mg) and Vitamin B₁₂ (10 μg).

### Example 4

A composition including elemental iron (175 mg), succinic acid (150 mg), ascorbic acid (200 mg), folic acid (1 mg) and Vitamin B₁₂ (10 μg).

### Example 5

A composition including elemental iron (225 mg), succinic acid (150 mg), ascorbic acid (200 mg), folic acid (1 mg) and Vitamin B₁₂ (10 μg).

### Example 6

A composition including elemental iron (250 mg), succinic acid (150 mg), ascorbic acid (200 mg), folic acid (1 mg) and Vitamin B₁₂ (10 μg).

### Example 7

A method for increasing iron absorption in a patient comprising administering to a patient in need thereof a composition or pharmaceutical kit including elemental iron (200 mg), ascorbic acid (200 mg), folic acid (1 mg) and Vitamin B₁₂ (10 μg) for days 1-10; administering a composition or pharmaceutical kit including elemental iron (200 mg), ascorbic acid (150 mg), succinic acid (150mg), folic acid (1 mg) and Vitamin B₁₂ (10 μg) for days 11-20; and administering succinic acid (300 mg) alone for days 21-28.

### Example 8

A method for increasing iron absorption in a patient comprising administering to a patient in need thereof a composition or pharmaceutical kit including elemental iron (200 mg), ascorbic acid (250 mg), folic acid (1 mg) and Vitamin B₁₂ (10 μg) for a first day; administering a composition or pharmaceutical kit including elemental iron (200 mg), ascorbic acid (100 mg), succinic acid (150mg), folic acid (1 mg) and Vitamin B₁₂ (10 μg) for a second day; and alternately administering the first and second day compositions or kits for a time effective to increase iron absorption in the patient.

1-42. (canceled)

43. A composition for increasing iron absorption in a patient, the composition comprising:
(a) a first iron promoter comprising a compound having Vitamin C activity; and
(b) a second iron promoter comprising an organic acid selected from the group consisting of succinic acid, acetic acid, citric acid, lactic acid, malic acid, glutamic acid, salts of succinic acid, salts of acetic acid, salts of citric acid, salts of lactic acid, salts of malic acid, salts of glutamic acid, derivatives of succinic acid, derivatives of acetic acid, derivatives of citric acid, derivatives of lactic acid, derivatives of malic acid, derivatives of glutamic acid, and combinations thereof;
wherein the first iron absorption promoter is formulated to dissolve in less than about 180 minutes following oral administration to the patient; and
wherein the second iron promoter is formulated for extended release such that less than substantially all of the second iron promoter dissolves within about 180 minutes following oral administration of the composition to a patient and substantially all of the second iron promoter dissolves in less than about 48 hours following oral administration of the composition to a patient,

44-49. (canceled)

50. The composition of claim 43, wherein the compound having Vitamin C activity is selected from the group consisting of L-ascorbic acid, calcium ascorbate, sodium ascorbate, magnesium ascorbate, potassium ascorbate, zinc ascorbate, L-threonic acid, L-xylonic acid and L-lyxonic acid.

51-52. (canceled)

53. The composition of claim 43, wherein the second iron absorption promoter comprises succinic acid.

54. The composition of claim 43, wherein the composition further comprises one or more forms of iron.

55. The composition of claim 43, wherein the one or more forms of iron are independently selected from the group consisting of carbonyl iron, chelated iron, soluble iron salts, slightly soluble iron salts, insoluble iron salts, chelated iron complexes and iron complexes.

56. The composition of claim 54, wherein the one or more forms of iron are selected from the group consisting of bis-glycine chelates of iron.

57-70. (canceled)

71. A method of increasing iron absorption in a patient, the method comprising administering at least two iron absorption promoters to a patient in need thereof, wherein a first of the iron absorption promoters is formulated for immediate release upon oral administration to the patient and a second of the iron absorptions promoters is formulated for extended release upon oral administration to the patient.

72. The method of claim 71, wherein the first and second iron absorption promoters are simultaneously administered to the patient.

73. The method of claim 71, wherein the first iron absorption promoter is formulated to dissolve in less than about 180 minutes following oral administration to the patient.

74. The method of claim 71, wherein the first iron absorption promoter is formulated to dissolve in less than about 20 minutes following oral administration to the patient.

75. The method of claim 71, wherein the second iron absorption promoter is formulated such that less than substantially all of the second promoter dissolves within about 180 minutes following oral administration to a patient, and substantially all of the second promoter dissolves in less than about 48 hours following oral administration to a patient.

76. The method of claim 71, wherein the second iron absorption promoter is formulated such that less than substantially all of the second promoter dissolves within about 8 hours following oral administration of the composition to a patient, and substantially all of the second promoter dissolves in less than about 24 hours following oral administration of the composition to a patient.

77. The method of claim 71, wherein the first iron absorption promoter comprises a compound having Vitamin C activity.

78. The method of claim 77, wherein the compound having Vitamin C activity is selected from the group consisting of L-ascorbic acid, calcium ascorbate, sodium ascorbate, magnesium ascorbate, potassium ascorbate, zinc ascorbate, L-threonic acid, L-xylonic acid and L-lyxonic acid.

79. The method of claim 71, wherein the first iron absorption promoter comprises ascorbic acid.

80. The method of claim 71, wherein the second iron absorption promoter comprises an organic acid selected from the group consisting of succinic acid, acetic acid, citric acid, lactic acid, malic acid, and glutamic acid.

81. The method of claim 71, wherein the second iron absorption promoter comprises succinic acid.

82. The method of claim 71, wherein at least one of the iron absorption promoters is selected to increase iron absorption within the intestinal lumen of the patient and at least one of the iron absorption promoters is selected to increase systemic iron absorption.

83. The method of claim 71, wherein the method further comprises administration of at least one source of iron to the patient.

84. The method of claim 83, wherein the at least one source of iron is selected from the group consisting of pharmaceutically acceptable iron compounds and metallic forms of iron.

85. The method of claim 83, wherein the at least one source of iron is selected from the group consisting of soluble iron salts, slightly soluble iron salts, insoluble iron salts, chelated iron, iron complexes, and non-reactive iron.

86. The method of claim 83, wherein the at least one source of iron comprises bis-glycine chelates of iron.

87. The method of claim 71, wherein the method further comprises administration of at least two iron compounds.

88. The method of claim 87, wherein the at least two iron compounds comprise at least one slow acting iron compound and at least one fast acting iron compound.
